(19) United States
(12) Patent Application Publication
(10) Pub. No.: US 2003/0108618 A1
(43) Pub. Date: Jun. 12, 2003

(54) Non-pharmacological method for treating depression, skin disorders, and improving overall health and wellness

(76) Inventor: Jack V. Smith, Arden, NC (US)

Correspondence Address: JACK V. SMITH, P.O. BOX 156, Arden, NC, 28704, US

(21) Appl. No.: 10/004,981

(22) Filed: Dec. 7, 2001

(51) Int. Cl. A61K 35/80; A61K 33/42; A61K 31/715; A61K 31/737

(52) U.S. Cl. 424/601; 424/195.17; 514/54

(57) ABSTRACT

The present invention is a method for manufacture and treatment of depression and health disorders using a non-pharmacologic stable negative ion solution.

### BACKGROUND OF THE INVENTION

1. Field of the Invention

The present invention is a method that relates to a liquid stable solution of negative ions (to be referred to as “ions”) and has been proven in research the ability to provide a non-pharmacologic therapy for the following health issues. Negative ions have been proven to have a positive affect on behavioral disorders (seasonal affective disorder (SAD)), relief of depression, relief from headaches, earaches, stuffy/snotty noses from hay-fever allergies and colds, for the treatment of asthma, bronchitis, thromboembolism, pain and peeling from sunburn and other bums, relief from itching from psoriasis, bug bites, and other skin irritations and improve memory, vitamin metabolism, sleep patterns and behavior. The ions solution can be applied directly, sprayed on, misted on skin, and can be inhaled. The ions solution can be placed in spray bottles, misters, humidifiers or other devices to disperse the solution into the air. The ions solution can also be used as a drink for internal intake.

It is therefore an object of the present invention to provide a non-pharmacologic method for treating health and behavioral issues such as SAD, depression, headaches, earaches, stuffy/snotty noses from hay-fever allergies and colds, asthma, bronchitis, thromboembolism, pain and peeling from sunburn and other burns, itching form psoriasis, bug bites, and other skin irritations and a method for improving memory, vitamin metabolism, sleep patterns and behavior by subjecting a patient to an exposure of high density negative ions directly using a liquid stable solution of negative ions via bathing, spray or mist aerosols, humidifier, mister, inhaler, drinking or other mode of application to the skin or internally through breathing or drinking.

A further object is to provide a method for the treatment of patients determined to have certain health and behavioral issues with exposure to high concentration of negative ions in a stable liquid matrix for a predetermined course of time.

2. Description of the Related Art

The present invention is a method that relates to a liquid stable solution of negative ions (to be referred to as “ions”) and negative ions have been demonstrated in numerous research papers and publications to have the ability to provide a non-pharmacologic therapy solution for many health and behavior issues. The present art can be applied directly, sprayed on, misted on skin, and can be inhaled. The ions solution can be placed in spray bottles, misters, humidifiers or other devices to disperse the solution into the air. The ions solution can also be used as a drink for internal intake.

There is no prior art that demonstrates, teaches or even alludes to a stable liquid solution of negative ions that can be used a for non-pharmacologic treatment for depression, behavioral disorders (seasonal affective disorder (SAD)), headaches, earaches, stuffy/snotty noses from hay-fever allergies and colds, asthma, bronchial asthma, and bronchitis, thromboembolism, pain and peeling from sunburn and other burns, itching form psoriasis, bug bites, and other skin irritations and improve memory, vitamin metabolism, sleep patterns and behavior.

About 13 million Americans suffer from winter depression at a fully syndromal level, and another approximate 30 million suffer a milder for of subsyndromal level and may not meet diagnostic severity criteria. There is prima facie evidence that reduced daylight availability in fall and winter is a likely precipitant of the depressive episode. It has been demonstrated, however not understood, that bright light can be an effective treatment for depression as well as negative ions.

It has been demonstrated that in addition to possibly having a profound effect on mood and energy, negative ions may have a strong impact on cognitive functioning. In 1965, in the journal “Psychophysiology”, a study, “Behavioral Effects of Ionized Air on Rats”, was published. The study showed that the effects of negative ions on the mental functioning of rats was tested. The researchers reported on page 358 of the journal that: “the water-maze performance improved 350%,” showing a dramatic improvement in cognitive functioning. Another article on negative ions published in April of 1978, in the science journal of “Ergonomics”, a study performed at the University of Surrey, England, entitled, “Air Ions and Human Performance”. Additionally, demonstrated that negative ions have a positive effect on cognitive functions. A quote from the article reads: “Three testing environments were used: natural, negative, and positive ionizations. Negative ionization was associated with a significant increment in performance as compared to controls.

Dr. Albert Kreuger, professor emeritus of the University of Calibfornia at Berkeley, performed research on negative ions in the 1950's. “Dr. Kreuger excited the scientific world when discovered ions to be biologically active, stimulated production of the powerful chemical serotonin (5-hydroxytryptamine). Present in many tissues (especially blood) and is a very active neuro-hormone which causes profound nueral, glandular, and digestive effects including stimulation of smooth muscles and nerves. It has been well published that serotonin may be important in controlling sleep.

In a 1995 issue of “Journal of Alternative and Comparative medicine”, a study was performed by Dr. Michael Terman (head of Columbia's Winter Depressions Department) and Dr. Jiuan Si Terman on the impact of negative ion therapy on people suffering from seasonal affective disorder (winter depression). It was a double blind study that produced remarkable results. “While a low negative ion generator provided little benefit, a high density negative ion generator gave relief from depression compatible to that given by Prozac and other antidepressants, without drug side effects.”

Dr. Bob Arnot performed a study published in the “Journal of Alternative and Complementary Medicine” and concluded that patients exposed to high-density negative ions had a significant relief of their symptoms for depression, almost identical to drugs, but without drug side effects. A patient under Dr. Arnot's care stated: “While I was on treatment, I felt excited, I felt energized. I felt alive.

A scientific article from the research papers of Bionic Products PTY., LTD., titled “The Treatment of Bronchial Asthma by Negative Aeroionisation” stated in the conclusion that “treatment with negative aeroions (therapeutic dose of 100 150 million aerions per session) leads to an improvement in the general state of the patients, a normalization of the blood picture, of the vascular and respiratory reactions, etc. Results of treatment: s) dyspnoeic attacks disappear in 55% of patients within 6 months; b) the intensity and number of attacks is reduced in 35% of patients; c) treatment is ineffective in 10% of cases. Negative aerions normalize the functional state of the central nervous system and in all cases the system of the patients with asthma.”

Another study published titled “Prevention of Post-Operative Thromboembloism by Negative Air lonisation in a Double-Blind Study” stated in the discussion “The fact that negative air ionization prevents thromboembolism cannot be denied.”

In 1966 at a hospital in Jersalem, doctors performed a series of tests on imfants with similar respiratory problems. They divided the infants into two groups. One group (the control group) no negative ion generator was used, and in another group a negative ion generator was used. The researchers reported that the negative ions without any other treatment (no drugs), seemed to cure attacks of asthma and bronchitis quicker than drugs, antibiotics included. They also noted no adverse side effect frequently found when treating children with drugs.

For more information concerning the beneficial effects of negative ions go to http://www.negativeions.com/scientificpublications.htm on the internet. The positive effect that the present art on the population and work force by improving overall health and well being cannot be denied. The use of the present device prevents aid in treatment of SAD, asthma, etc. It will save the users (patients) millions in medical bills and companies millions of hours not lost because of employee calling in because they do not feel good. This invention will have a positive effect on the economy to the tune of millions of dollars.

There is no prior that produces the unexpected results of the present device and the answers to a solution to that was never before even recognized that the present art provides. The prior art teaches away from the present art in that it goes in a completely different direction. That is to say that the negative ion generators of the past which are machines that produce negative ions (not a liquid solution of negative ions) were not designed to be used to apply directly to the skin, to be used as drink, etc., but strictly for the production of negative ions. The present device when placed in a humidifier produces 2.5 times the concentration of negative ions than a commercially available ion generator machine does.

A thorough search of patents, publications, and research revealed no relative art (i.e., prior art) showing any direct correlation to this novel invention. The search included the USPTO (United States Patent Office) data base with no patents issued for a device that is a liquid stable solution of negative ions that can be used for non-pharmacological treatment of multiple health and behavior issues.

### SUMMARY OF THE INVENTION

The present invention is a method designed to advance the art of treatment for health and behavioral issues. This method relates to a liquid stable solution of negative ions (to be referred to as “ions”). As previously discussed, negative ions have been demonstrated in numerous research papers and publications to have the ability to provide a non-pharmacologic therapy solution for many health and behavior issues. The present art can be applied directly, sprayed on, misted on skin, and can be inhaled. The ions solution can be placed in spray bottles, misters, humidifiers or other devices to disperse the solution into the air. The ions solution can also be used as a drink for internal intake.

There is no prior art that demonstrates, teaches or even alludes to a stable liquid solution of negative ions that can be used a for non-pharmacologic treatment for depression, behavioral disorders (seasonal affective disorder (SAD)), relief of headaches, earaches, stuffy/snotty noses from hay-fever allergies and colds, relief of asthma, bronchial asthma, and bronchitis, thromboembolism, pain and peeling from sunburn and other burns, relief of itching form psoriasis, bug bites, and other skin irritations and improve memory, vitamin metabolism, sleep patterns and behavior.

The present art allows the use of a stable solution of negative ions never before discovered to aid in the treatment and prevention of disease. This art can be used without the use of electricity or other machines to effect it ability to treat. The ions solution can be placed in spray bottles, inhalers, misters or applied directly in the form of a bath of shower. The present art allows the user the ability to affect treatment without the need for machines or power remotely. It will save the patient (user) money in the fact no additional device is required to affect treatment. In remote, third world countries, the solution could be used where power and machines are not available. This is not to say that the present art cannot use machines to affect it treatment. The use of humidifiers and other devices that will allow the dispersal of the ions solution into the air or onto a patients skin via a motorized inhaler, powered sprayer, or other such devices are a distinct ability of the present device to affect treatment.

Correspondingly, another advantage of the present art is to provide a stable solution that can be drank to affect the ingestion of negative ions. This is truly a one step process which is not currently known in the art.

In addition the present art provides for a unique method to increase the amount of negative ions into the air while increasing the moisture (humidity) of a room making it easier to breath in the aerosols of negative ions.

It has been found that the foregoing objects of the present art are accomplished in accordance with this invention by providing a stable solution of negative ions that can be immediately provided to the patient in a variety of methods.

The present invention provides a non-pharmacological method of treatment for health and behavorial problems in a human subject using a negative ion solution, and the method being characterized by the following steps:

a) Exposing the subject to negative ions by contacting the subject with the negative ion solution in the form of spray, mist, or liquid; and

b) During exposure the negative ion solution comes into contact with the subjects skin, lungs, mouth or other parts of the subjects body to affect said treatment.

c) The said treatment occurs without the use of a negative ion generator.

Other aspects and advantages of the present invention appear more clearly from reading the following detailed description of the preferred embodiment of the invention, given by way of example and made with reference to the accompanying tables. Such as the determination of exactly how the device works. A thorough search of the literature reveals no relative art resembling this technology; therefore, this invention is clearly a novel in creation, and is not obvious to anyone skilled in the art, in fact the prior art devices teaches away from the present art in that the prior art requires a negative ion generator device be used (this is not a requirement of the present device in fact the present device is a teaching of simplicity with no manipulation or essential need for electricity as a requirement) and the prior art devices teach away from the present device in that the prior art requires a generator which obviously requires a electricity to affect treatment, and the prior art teaches away from the present device in that the prior teaches the use and requirement of a negative ion generator which is expensive. There are certain aspects of the present art that can be found in the prior art (e.g. the use of negative ions) but no prior has advanced the art of non-pharmacological treatment for health and behavioral problems and diseases. This art solves an unrecognized problem that was never before even recognized. Specifically this allows for the user the unexpected results of using a device that is simple, efficient and cost effective that only utilizes a spray bottle, or inhaler or other simple device to affect treatment, etc., for a much more effective, economical and practical method of treatment of depression, behavioral disorders (seasonal affective disorder (SAD)), headaches, earaches, stuffy/snotty noses from hay-fever allergies and colds, asthma, bronchial asthma, and bronchitis, thromboembolism, pain and peeling from sunburn and other burns, itching form psoriasis, bug bites, and other skin irritations and improve memory, vitamin metabolism, sleep patterns and behavior.

Therefore with the present invention it is very important that the ability to affect treatment without the use of a negative ion generator, electricity, or other machines virtually eliminates a problem or inability of the prior art.

### DETAILED DESCRIPTION OF THE PREFERRED EMBODIMENTS

The present invention will now be described more fully with reference to the accompanying tables, in which the preferred embodiments of the present art invention efficacy are shown. It is understood from the embodiments that a person skilled in the art may make variations and modifications without departing from the spirit and scope of the invention. Such as changing the formula's various components amounts, concentrations, or using similar but functional compounds while remaining within the scope and spirit of the invention.

The present invention is a method designed to advance the art of treatment for health and behavioral issues. This method relates to a liquid stable solution of negative ions (to be referred to as “ions”). Negative ions have been demonstrated in numerous research papers and publications to have the ability to provide a non-pharmacologic therapy solution for many health and behavior issues. The present art can be applied directly, sprayed on, misted on skin, and can be inhaled. The ions solution can be placed in spray bottles, misters, humidifiers or other devices to disperse the solution into the air. The ions solution can also be used as a drink for internal intake.

The present invention is a stable liquid solution of negative ions that can be used a for non-pharmacologic treatment for depression, behavioral disorders (seasonal affective disorder (SAD)), relief of headaches, earaches, stuffy/snotty noses from hay-fever allergies and colds, relief of asthma, bronchial asthma, and bronchitis, thromboembolism, pain and peeling from sunburn and other burns, relief of itching form psoriasis, bug bites, and other skin irritations and improve memory, vitamin metabolism, sleep patterns and behavior without the use of a negative ion generator.

The present art allows the use of a stable solution of negative ions never before discovered to aid in the treatment and prevention of disease. This art can be used without the use of electricity or other machines to effect it ability to treat. The ions solution can be placed in spray bottles, inhalers, misters or applied directly in the form of a bath of shower. The present art allows the user the ability to affect treatment without the need for machines or power remotely.

TABLE 1
METHOD COMPARISON
Measurements (ions/cm³)

| Readings¹: | Neg Ion Generator² | Neg Ion Generator³ | Humidifier⁴ |
|---|---|---|---|
| 1 | −1300 | −1000 | −3000 |
| 2 | −1200 | −700 | −2800 |
| 3 | −1000 | −1200 | −3500 |
| Mean: | −1167 | −967 | −3100 |

¹The readings were taken using a “Air Ion Counter” from AlphaLab, Inc.
²Negative Ion Generator from Comtech Research Model IG-133.
³Negative Ion Generator Model K-3000.
⁴Sunbeam's 2 Gallon Cool Mist Humidifier containing the negative ion solution.

The study was conducted in an 18′×20′ room over a three-day period.

Conclusion of data from Table 1. The data clearly shows that the negative ion solution of the present invention produced more negative ions than 2 of the prior arts negative ion generators by a factor of 2.5 times without the use of a negative ion generator.

It is has been well published that negative ions affect serotonin levels in humans. It is also known that serotonin has been associated with and plays an important role in sleeping. In addition, negative ions have been demonstrated extensively to have positive affects on individuals suffering from depression and other behavioral disorders.

TABLE 2
Sleep Affect from Negative Ions Exposure

| Interpretive | | |
|---|---|---|
| Measurements | | |
| Subject(s) | | |
| Exposure¹: | No Humidifier | Humidifier² |
| 1 | No Noticeable affect | Sleep soundly without wakening |
| 2 | No Noticeable affect | Sleep soundly without wakening |
| 3 | No Noticeable affect | Sleep soundly (woke only once) |

¹The different subject(s) were exposed to nightly sleep periods (typically 8 hours) with and without a humidifier.
²Sunbeam's 2 Gallon Cool Mist Humidifier containing the negative ion solution.

Conclusion of data from Table 2. The data clearly shows that the humidifier containing the negative ion solution of the present invention produced a positive affect for sound sleep versus no humidifier with negative ion solution present. Although data not presented this study was done in blind fashion with some subjects having no negative ion solution in there generators. In those subjects without the negative ion solution in their humidifiers (only water) there was no noticeable difference in their sleep pattern.

TABLE 3
Affect on Severe Colds, Stuffiness, and Earaches
from Negative Ions Exposure Interpretive Measurements

| Subject(s) | | |
|---|---|---|
| Exposure¹: | No Humidifier | Humidifier(s)² |
| 1 | No Noticeable affect | Cleared up cold and sleep soundly |
| 2 | No Noticeable affect | Cleared/Stopped earache |
| 3 | No Noticeable affect | Cleared Stuffy nose and tiredness |

¹The different subject(s) ages from 14 to 73 were exposed to nightly sleep periods (typically 8 hours) with and without a humidifier.
²Humidifier(s) containing the negative ion solution.

Conclusion of data from Table 3. The data clearly shows that the humidifier containing the negative ion solution of the present invention produced a positive affect for clearing up colds, earaches, and stuffiness versus no humidifier with negative ion solution present.

TABLE 4
Affect on Asthma and Bronchitis from Negative Ions Exposure
Interpretive Measurements

| Subject(s) | | |
|---|---|---|
| Exposure¹: | No Spray | Spray Bottle² |
| 1 | No Noticeable affect | No coughing for two weeks after |
| 2 | No Noticeable affect | Stopped attacks upon use |
| 3 | No Noticeable affect | No more episodes of asthma attacks |

¹The different subject(s) were exposed to a spray bottle of negative ion solution spray directly into the mouth.
²Spray bottle containing the negative ion solution.

Conclusion of data from Table 4. The data clearly shows that the spray bottle containing the negative ion solution of the present invention produced a positive affect in regards to asthma, coughing and bronchitis like syndromes. As supported by the multitude of studies this data demonstrates the positive affect that negative ions can have on asthma and bronchitis. It may be noted that the subject(s) of the study stated that there was no need for an inhaler or other treatments for asthma while this study was being conducted.

TABLE 5
Affect on Sunburn, itching and other Burns from Negative Ions Exposure
Interpretive Measurements

| Subject(s) | | |
|---|---|---|
| Exposure¹: | Spray Bottle² | Spray Bottle³ |
| 1 | No Noticeable affect | Immediate Relief/No peeling |
| 2 | No Noticeable affect | No peeling |
| 3 | No Noticeable affect | Immediate Relief/No blister |
| 4 | No Noticeable affect | Immediate Relief/No blister |
| 5 | No Noticeable affect | Immediate Relief/No peeling |
| 6 | Continued Itching | Immediate Relief/No itching |
| 7 | Continued Itching | Immediate Relief/No icthing |

¹The different subject(s) were exposed to a spray bottle of negative ion solution spray directly onto the skin or area of exposure to sun, poison ivy, or stove top.
²Spray bottle containing water.
³Spray bottle containing negative ion solution.

Conclusion of data from Table 5. The data clearly shows that the spray bottle containing the negative ion solution of the present invention produced a positive affect in regards to relieving suffering from sunburn/peeling, oven burns/blisters, and itching from poison ivy.

As illustrated in the data from the tables the present art provides for a unique method to increase the amount of negative ions into the air while increasing the moisture (humidity) of a room making it easier to breath in the aerosols of negative ions.

It has been found that the foregoing objects of the present art are accomplished in accordance with this invention by providing a stable solution of negative ions that can be immediately provided to the patient in a variety of methods.

The present invention provides a non-pharmacological method of treatment for health and behavorial problems in a human subject using a negative ion solution, and the method being characterized by the following steps:

a) Exposing the subject to negative ions by contacting the subject with the negative ion solution in the form of spray, mist, or liquid; and

b) During exposure the negative ion solution comes into contact with the subjects skin, lungs, mouth or other parts of the subjects body to affect said treatment.

c) The said treatment occurs without the use of a negative ion generator.

It should be noted that the discovery of the present invention was surprising and led to unexpected results in that it produced a stable solution of negative ions.

The following examples are provided to further illustrate the inventive aspects of the present discovery, and to further describe preferred embodiments. As such, they are intended as being merely illustrative, and are not to be construed as limiting the scope of the claims appended hereto.

### EXAMPLE 1

The following procedure is a method for manufacturing a liquid stable solution of negative ions that can be placed in an humidifier, a spray bottle, mister, inhaler, used as a drink, used to shower with, or applied to the skin or body in some other manner or fashion and still remain within the spirit and scope of the invention.

The liquid stable solution of negative ions is successively manufactured as follows:

1. Obtain 1 liter of distilled or deionized water water.

2. Add 0.05 g polyphosphate (negative ion active ingredient) to 1 liter of water.

3. Mix until dissolved.

Once the above solution is completely dissolved in liquid then store in a clean dry container. Test the solution for negative ion content. Do this by measuring the Zeta Potential (mV) which should be greater than ⁻10.0 mV (eg. the higher the ⁻mV reading the higher the negative ion concentration, that is to say that ⁻50.0 mV is a higher negative ion concentration than ⁻10.0 mV). For use, take the above solution and place in a spray bottle, humidifier, or other device to so that the subject can be exposed to a negative ion solution, and the method being characterized by the following steps:

a) Exposing the subject to negative ions by contacting the subject with the negative ion solution in the form of spray, mist, or liquid; and

b) During exposure the negative ion solution comes into contact with the subjects skin, lungs, mouth or other parts of the subjects body to affect said treatment.

c) The said treatment occurs without the use of a negative ion generator.

It should be noted that the discovery of the present invention was surprising and led to unexpected results in that it produced a stable solution of negative ions. The negative ion active ingredient polyphosphate used in example 1 may be substituted with one or more selected from the group consisting of: sodium tripolyphosphate, pentasodium triphosphate, carrageenan, irish moss extracts, sea weed extracts, red seaweed extracts, beta-1,3-Dgalactose and alpha-1,4-3,6-anhydro-D-galactose polysaccharides, phycocolloids, sodium carrageenan, potassium carrageenan, calcium carrageenan, or a salt form of carrageenan, extracts of kelp, extracts of alaria, and extracts of laver-nori.

Note the above could additionally be substituted with any other chemical or compound that can produce a stable high negative ion concentration in a liquid and be used as defined by the present art and still fall within the spirit and scope of the present invention.

### EXAMPLE 2

The following procedure is a method for manufacturing a liquid stable solution of negative ions that can be placed in an humidifier, a spray bottle, mister, inhaler, used as a drink, used to shower with, or applied to the skin or body in some other manner or fashion and still remain within the spirit and scope of the invention.

The liquid stable solution of negative ions is successively manufactured as follows:

1. Obtain 1 liter of distilled or deionized water water.

2. Add 0.05 g carrageenan (negative ion active ingredient) to 1 liter of water.

3. Mix until dissolved.

Once the above solution is completely dissolved in liquid then store in a clean dry container. Test the solution for negative ion content. Do this by measuring the Zeta Potential (mV) which should be greater than ⁻10.0 mV (eg. the higher the ⁻mV reading the higher the negative ion concentration, that is to say that ⁻50.0 mV is a higher negative ion concentration than ⁻10.0 mV). For use, take the above solution and place in a spray bottle, humidifier, or other device to so that the subject can be exposed to a negative ion solution, and the method being characterized by the following steps:

d) Exposing the subject to negative ions by contacting the subject with the negative ion solution in the form of spray, mist, or liquid; and

e) During exposure the negative ion solution comes into contact with the subjects skin, lungs, mouth or other parts of the subjects body to affect said treatment.

f) The said treatment occurs without the use of a negative ion generator.

It should be noted that the discovery of the present invention was surprising and led to unexpected results in that it produced a stable solution of negative ions. The negative ion active ingredient carageenan used in example 2 may be substituted with one or more selected from the group consisting of: polyphosphate, sodium tripolyphosphate, pentasodium triphosphate, irish moss extracts, sea weed extracts, red seaweed extracts, beta-1,3-Dgalactose and alpha-1,4-3,6-anhydro-D-galactose polysaccharides, phycocolloids, sodium carrageenan, potassium carrageenan, calcium carrageenan, or a salt form of carrageenan, extracts of kelp, extracts of alaria, and extracts of laver-nori.

Note the above could additionally be substituted with any other chemical or compound that can produce a stable high negative ion concentration in a liquid and be used as defined by the present art and still fall within the spirit and scope of the present invention.

The following is a brief exlanation of the novel present art. It is a non-pharmacological method of treatment for health and behavorial problems in a human subject using a negative ion solution comprising the steps of producing a negative ion solution with a zeta ion potential of at least −10 mV or greater by successively dissolving a negative ion active ingredient completely in water, and; exposing the subject to negative ions by contacting the subject with the negative ion solution in the form of spray, mist, humidifier, inhaler, liquid drink or shower without the use of a negative ion generator. As taught the negative ion active ingredient can be selected from the group consisting of polyphosphate, sodium tripolyphosphate, pentasodium triphosphate, carrageenan, irish moss extracts, sea weed extracts, red seaweed extracts, beta-1,3-Dgalactose and alpha-1,4-3,6-anhydro-D-galactose polysaccharides, phycocolloids, sodium carrageenan, potassium carrageenan, calcium carrageenan, or a salt form of carrageenan, extracts of kelp, extracts of alaria, or extracts of laver-nori. It is also understood that the water used in the prior examples can be substituted with other suitable solvents selected from the group consisting of alcohol, buffers, or organics solvents. And of course present art when using a spray, mist, inhaler, liquid drink or shower does not require electricity.

This invention is going to save the general public, government and private enterprise millions of dollars in save time and labor. It is a well known fact that millions of dollars are spent daily on health care and preventive medicine for symptoms of flu and cold, depression, etc. The use of the present invention will make for a healthier world The invention has been described in detail with particular reference to a preferred embodiment and the operation thereof and it is understood that variations, modifications, and substitution of equivalent means can be effected and still remain within the spirit and scope of the invention. And all such modifications and variations are to be included within the scope of the invention as defined in the appended claims.

That which is claimed is:

1. A non-pharmacological method of treatment for health and behavorial problems in a human subject using a negative ion solution comprising the steps of:
a) Producing a negative ion solution with a zeta ion potential of at least −10 mV or greater by successively dissolving a negative ion active ingredient completely in water, and;
b) Exposing the subject to negative ions by contacting the subject with the negative ion solution in the form of spray, mist, humidifier, inhaler, liquid drink or shower without the use of a negative ion generator.

2. The method according to claim 1 wherein said negative ion active ingredient is selected from the group consisting of polyphosphate, sodium tripolyphosphate, pentasodium triphosphate, carrageenan, irish moss extracts, sea weed extracts, red seaweed extracts, beta-1,3-Dgalactose and alpha-1,4-3,6-anhydro-D-galactose polysaccharides, phycocolloids, sodium carrageenan, potassium carrageenan, calcium carrageenan, or a salt form of carrageenan, extracts of kelp, extracts of alaria, or extracts of laver-nori.

3. The method according to claim 1 wherein said water may be substituted with other suitable solvents selected from the group consisting of alcohol, buffers, or organics solvents.

4. The method according to claim 1 using a spray, mist, inhaler, liquid drink or shower does not require electricity.
